(19) United States
(12) Patent Application Publication
(10) Pub. No.: US 2018/0360362 A1
(43) Pub. Date: Dec. 20, 2018

(54) WEARABLE AND/OR PORTABLE DEVICES

(71) Applicants: Natalia SARKISIANS, Oakland, CA (US); Stanislav SARKISYANTS, Oakland, CA (US)

(72) Inventors: Natalia SARKISIANS, Oakland, CA (US); Stanislav SARKISYANTS, Oakland, CA (US)

(21) Appl. No.: 16/011,046

(22) Filed: Jun. 18, 2018

Related U.S. Application Data
(60) Provisional application No. 62/521,908, filed on Jun. 19, 2017.

(51) Int. Cl. A61B 5/15; A61B 5/145; G16H 20/17; A61B 5/151; A61B 5/00

(52) U.S. Cl. CPC A61B 5/150419; A61B 5/14503; A61B 5/1411; A61B 5/15142; A61B 5/6801; G16H 20/17

(57) ABSTRACT

Provided herein are wearable and/or portable devices for the detection of at least one analyte in a body fluid. In accordance with certain aspects of the present invention, there are also provided methods for the use of invention devices for the automated administration of essential vitamins, minerals or other nutrients to a subject in need thereof. In accordance with the present invention, there are also provided body fluid sampling devices. In certain aspects of the present invention, there are also provided wearable and/or portable devices for the detection of at least one analyte in a body fluid, and the automated administration of essential vitamins, minerals or other nutrients to a subject determined to be in need thereof based on the results provided by the means for detection.

### CROSS-REFERENCE TO RELATED APPLICATIONS

This application claims the benefit of U.S. Provisional Application No. 62/521,908, filed Jun. 19, 2017, which is hereby incorporated by reference, in its entirety and for all purposes.

### FIELD OF THE INVENTION

The present invention relates to wearable and/or portable devices for the detection of at least one analyte in a body fluid. In certain embodiments, the present invention relates to body fluid (blood) sampling devices. In certain embodiments, the present invention relates to methods for the automated administration of defined nutrients to a subject in need thereof.

### BACKGROUND OF THE INVENTION

The information provided herein and references cited are provided solely to assist the understanding of the reader, and does not constitute an admission that any of the references or information is prior art to the present invention.

The regular consumption of vitamins and minerals is necessary for proper regulation of the body's metabolic functions. Significant research, which is still ongoing, has presently amassed a good deal of knowledge as to the type and quantity of vitamins and minerals necessary for good health, and how various individual-specific factors including age, gender, whether a woman is pregnant or nursing, general health, and a person's typical daily diet affect their daily intake requirement of vitamins and minerals. Also, certain diseases can affect a person's daily vitamin and mineral requirements.

Fortunately, essential vitamins and minerals are found in various foods. However, certain diets may be lacking in certain vitamins or nutrients and, as mentioned above, certain factors specific to each individual can vary these needs. These individual factors may affect average daily vitamin and mineral needs over a short period of time, perhaps even causing a variance from day to day, or they may affect a person's long-term vitamin and mineral daily intake needs.

For example, some people may require additional vitamins and mineral supplements due to injury, disease, impaired immune response, the effects of aging, and the like. Additionally, athletes and health-conscious people may wish to ensure that they are receiving ample quantities of vitamins and minerals. Similarly, those with a large daily expenditure of calories may require increased amounts of certain vitamins and minerals.

For all of these and other reasons the use of vitamin and mineral supplements is widespread. A recommended daily intake of vitamins and minerals is published. However, due to specific individual needs, certain users may wish to moderate the quantity of vitamin and mineral supplements they are consuming to values other than those contained in any available recommended daily intake publication.

Most people, though, will benefit when they take care to consume the recommended daily value of all essential vitamins and minerals.

As essential vitamins and minerals help to control and modulate metabolic functioning they are also referred to herein as “bioactive” or as “bio-active” agents. As used herein, the term “nutraceutical” refers to a branch of commerce that pertains to the development, manufacture, and sale of vitamin, mineral, and general nutritional supplements.

The use of supplements to support or improve health is well known. As used herein, the term “supplements” includes vitamins, minerals as well as any other agent, other than food, that is consumed with the intention of augmenting health. For example, certain herbs, mushrooms, berries and other substances including royal jelly, various acids, and plant or animal extracts may also be used as bioactive supplements.

Vitamins and minerals are the most common and widely available types of supplements; indeed, these materials are taken by many people on a regular basis. The most common regimen is to take various supplements at the same time each day, usually in the morning. This is especially true, for example, when taking a multi-vitamin tablet.

Nutritional standards have been established by various sources that define the minimum daily intake of vitamins and minerals necessary for maintaining or supporting good health. When accomplished, overt symptoms of vitamin and mineral deficiency are avoided. The standard established by the United States Food and Drug Administration (FDA) defines “Daily Value” levels or more simply, “Daily Values” for vitamins and minerals.

However, there are two significant problems associated with the use of supplements. The first problem is that certain supplement compounds are potentially toxic if consumed in excess quantity, and the second problem is that many supplement compounds interact with one-another when consumed together. The interaction that occurs may either contribute to or detract from their beneficial absorption into the body.

Over-the-counter vitamin and mineral supplements fail to consider both possibilities.

Unfortunately, with regard to excess quantity, in our culture and many others the belief is that “more is better”. As a result many vitamin and mineral supplements have evolved over time to include many times the recommended daily norm (value) for certain ingredients. While a small vitamin and mineral excess is easily tolerated by most people, very high quantities are to be avoided (unless recommended by a health care practitioner for a specific illness or condition).

The reason why many vitamins include an excess is twofold. First, people may see two products for sale where a first includes 100% of the daily recommended amount of a particular vitamin and where a second includes 200% (or more) of the daily recommended amount. An uninformed populace is likely to choose the 200% (or more) version, believing that the extra quantity certainly cannot harm them and perhaps hoping that it just might be of some additional benefit. They might reflect at the time of purchase, “If more wasn't better than less, then manufacturers certainly wouldn't include more than the recommended amount.”

In view of the continuation of this trend for many years, it is not difficult to understand why, today, certain manufacturers offer supplements that contain, for example, 3333% of the daily required amount of certain vitamins (e.g., vitamin B1 (Thiamin)). The uninformed populace continues to wrongly believe that consumption of an excess quantity of supplement ingredients will cause no harm and is, therefore, to their advantage. Consequently, “more” of anything tends to help selling the product, especially when the cost of competitive products is comparable.

Additionally, many people are willing to pay considerably more for supplements that include a significant excess of ingredients above recommended daily values, believing them to be more effective. In reality, the opposite may be closer to the truth.

The FDA has unwittingly added to the misconception that more is better with regards to vitamins and minerals, by emphasizing the risks of insufficient intake levels. As a result, the population is now generally well-aware that too little of certain vitamins and minerals is detrimental to health while simultaneously remaining largely unaware that too much can also negatively affect one's health.

Simply put, the body must process all that is ingested and must excrete that which becomes waste and excess. An excess of vitamins and minerals (i.e., supplement ingredients) places additional stress on internal organs. When consuming supplements with much higher dosages than are considered necessary, it also subjects the entire body to unnaturally high exposure levels of certain vitamins and minerals. Nearly every cell of the body is then subjected to these high exposure levels as the nutrients (i.e., the vitamins and minerals) are carried in the bloodstream. This bathes the cells with unnaturally high supplement levels.

While a limited amount of data is already known regarding these adverse effects, the long-term effect of exposure to above-normal concentrations of vitamins and minerals (when considering either individual ingredients or when considering multiple ingredients in concert with one another) is not completely understood. Different types of cells may exhibit a greater or lesser tolerance over the course of time. Much more research is still required at both the cellular and the systemic level to understand these effects better.

Since nutritional supplementation is a relatively new science, it must be acknowledged that our current level of understanding, is, in many ways, still incomplete.

Additionally, high concentrations of vitamins and mineral tend to accumulate in particular organs, thereby subjecting them to unnaturally high, and potentially toxic levels. Damage, including potentially irreversible harm, is possible.

Consequently, the crux of the prior-art vitamin consumption problem can be appreciated when one reflects on the fact that vitamins and minerals are necessary to support life, but consumption of an excess quantity of vitamins and/or minerals may actually be harmful.

Accordingly, recent research regarding the long-term efficacy and safety of multivitamin supplements may suggest that there is no actual benefit with regards to increasing life expectancy or reducing the potential for developing or contracting disease. Worse yet, some studies suggest that the taking of vitamins may actually increase the possibility of early death or disease.

It must be understood that such data is derived from studies taken in a culture where excess vitamin intake is commonplace. Therefore, adverse effects make sense. Yet, a proper intake of vitamins and minerals is essential to life, and when properly accomplished must improve longevity and health as compared to a vitamin and mineral deficient diet.

Furthermore, there may be adverse synergistic effects that occur on a system-wide basis which are not presently well understood. For example, an organ that is exposed to a toxic level of a vitamin or mineral may adversely affect body chemistry or metabolic function which, in turn, may affect the functioning or health of other organs, the immune system, and even the brain. The compound effect when multiple organs are simultaneously exposed to high exposure levels of vitamins and minerals is not well understood.

Impaired body performance, low energy, loss of mental acuity and increased susceptibility to various diseases may occur as a result of the over-consumption or inappropriate formulation of vitamins and minerals. Demonstrating a clear scientific correlation to these and other possible detrimental health effects may require years or even decades of data to establish. In the meantime, the health of many people may suffer needlessly.

As alluded to earlier, preliminary research data has revealed a spike in cancerous diseases that appears to correlate with consumption of supplements, which amplifies the above concerns.

It is also important to note that certain nutrients are water soluble while others are fat (oil) soluble. Water soluble ingredients tend to be easier to excrete from the body whereas fat soluble ingredients are more likely to accumulate to excessive and even toxic levels. For example, certain fat-soluble minerals such as iron, if consumed in excess, may cause or contribute to the onset of certain cancers (i.e., they may be carcinogenic).

One thing is becoming clearer in nutritional science and that is that more is not necessarily better when it comes to vitamin and mineral supplements. Based on current data and prudent thought it is safe to state that satisfying the Daily Value needs without protracted, significant excess intake is preferable.

Prior art supplements have not yet provided an ideal solution regarding long-term daily vitamin and mineral intake, and so excess quantities are at times consumed by many people. One of the reasons for excess quantities is as described above, to persuade consumers that a particular product is superior to another because it includes a greater quantity of one or more ingredients.

Sometimes, however, excess quantities can occur in response to newly published research data that seems to suggest that a greater quantity than previously understood of some particular supplement ingredient is either necessary to promoting health or is desirable. For example, new data may seem to suggest that a higher dose of some particular vitamin or mineral may function as a prophylactic in helping to prevent certain adverse health effects from occurring. This can spur manufacturers to introduce the ingredient as a new, additional supplement ingredient or, if the ingredient is already included in a supplement, to ramp up the quantity of that particular ingredient included in currently available supplement offerings.

The manufacturer would then, in all likelihood, include a highly visible indication of the (new) ingredient and the higher dose on the packaging to promote sales. In this manner, a new supplement “fad” may be created.

But there is another reason why certain manufacturers include higher doses of certain ingredients, and that is because of adverse interactions that may occur between certain ingredients. This includes the topic of compatibility, or lack thereof, between vitamins and/or minerals present in a supplement formulation. As mentioned above, many manufacturers provide a daily multi-vitamin and/or a multi-mineral (commonly referred to as a “multivitamin” supplement) that includes a large quantity of a variety of different ingredients, preferably all that are believed to be necessary to support and/or promote good health on a daily basis. The reason for doing so is clear: to satisfy consumer demand for a “one pill does all” solution, it is preferable to provide a single dose supplement for daily consumption that includes all essential vitamins and minerals.

However, consuming all your nutrition in a single dose is not possible due to negative interactions that occur between certain supplement ingredients. If any two (or more) ingredients when taken together produce a negative interaction that limits absorption or efficacy of any ingredient, a first prior art solution has simply been to significantly increase the dose of one or more of the ingredients.

This increase in quantity is accomplished with the hope or expectation that after all negative interactions between ingredients have occurred, there will still remain a sufficient quantity of each of the ingredients for absorption and beneficial use. However, the efficacy of this approach is unclear and, as recent research may suggest, is detrimental to longevity and good health.

Due to the general lack of awareness regarding optimal daily dosing of vitamins and minerals, much of the general population does not question the reason behind the large ingredient quantities that so often occur in many of today's supplements.

With further regard to interactions that occur between ingredients in a supplement, an interaction can be either positive or negative. Positive interactions between ingredients promote absorption and assimilation of essential nutrients and are instances of positive compatibility. Negative interactions between ingredients retard, limit, or prevent absorption and assimilation of essential nutrients and are instances of negative compatibility.

Research has confirmed that certain supplement ingredients, when two or more are present in the same supplement and simultaneously ingested, will diminish or even annul the absorption and assimilation of one or both of the ingredients due to chemical reactions and interaction with bodily processes. It is desirable, then, to not include these ingredients in the same supplement or to retard their release to avoid negative interactions that could interfere with proper absorption and assimilation of any of the essential nutrients.

For example, when a supplement containing iron is mixed with certain other supplement ingredients, such as the mineral calcium, a decrease in the absorption of iron into the body occurs as well as a decrease in the absorption of calcium. This is an example of a negative interaction or negative compatibility between these ingredients.

However, when a supplement containing iron is mixed with a certain other different supplement ingredient, such as vitamin C, an increase in the absorption of iron into the body occurs. This illustrates a positive interaction or positive compatibility between these supplement ingredients.

Therefore, it is logically desirable to include vitamin C in the supplement that includes iron while avoiding the inclusion of calcium in the same supplement.

Delayed-release coatings have been employed as a second possible prior-art solution to delay the release of certain supplement ingredients and, thereby, hopefully avoid negative interaction between certain of the ingredients. The goal is to delay release and subsequent absorption of one or more of the ingredients in a supplement until after a sufficient quantity of a potentially conflicting ingredient (i.e., an ingredient that would cause a negative interaction to occur if simultaneously exposed to each other) had already been absorbed into and assimilated by the body.

An example of a prior-art solution might be to include uncoated iron and uncoated vitamin C with calcium that included a delayed-release coating. The initial exposure of the iron with the vitamin C would, in theory, produce a positive interaction to increase iron absorption. The delayed release of the calcium would, in theory, avoid the negative interaction between the iron and the calcium.

However, coatings may not provide a long-enough time delay (i.e., several hours) to sufficiently prevent negative interaction from occurring. If a coating were included in a supplement that provided a sufficiently long time delay prior to release of the coated ingredient for absorption, the ingredient may have passed too far along the gastrointestinal tract to permit adequate absorption and assimilation. Therefore, it may not be possible to avoid negative interactions between supplement ingredients by reliance entirely upon the use of delayed-release coatings being applied over certain (incompatible) supplement ingredients.

Additionally, another reason why the use of delayed release coatings that are applied over certain supplement ingredients may not be used with combination multi-vitamin and mineral supplements is because the general public does not know enough about supplement interactions to seek products that avoid negative interactions. In other words, for the most part the general population does not currently demand the use of delayed-release coatings as a method of helping to avoid or limit negative interactions between supplement ingredients.

Also, because the long-standing trend has been to include increasingly high doses of many of the ingredients in most vitamin/mineral supplements, the resulting tablet size has also grown over the course of time. Many supplements have already become so large that some people have difficulty swallowing them. If delayed-release coatings were also used while maintaining the same wide-spectrum array of nutrients and the same high dosing levels of the nutrients, the size of the multi-vitamin might potentially increase beyond that which could be comfortably swallowed by many people.

Another deterrent to the use of delayed-release coatings is that the manufacturing process is made more complex and time-consuming by their inclusion. Also, additional materials (i.e., the material used to form the coatings) must be included. Increased manufacturing complexity, time and increased materials' cost combine to further increase supplement cost whenever coatings are utilized.

The prior-art use of delayed-release coatings also includes their use with certain prescription and over-the-counter medicines. The goal is to provide a progressive release of the medicine in order to limit initial peak bloodstream levels and thereby provide a more consistent bloodstream level over a longer course of time. Coatings can provide an improved therapeutic benefit over a longer duration of time by better controlling bloodstream levels of prescription and over-the-counter medications. However, the use of delayed-release coating in supplements is generally not preferred if they can be avoided.

Additionally, currently available supplements are geared to mass needs. It has been difficult to determine how to vary supplement ingredients for the vast array of individual differences that may occur. As such, many of the currently available supplements tend to ignore individual factors, such as gender, general physical health, activity level, age, typical daily diet, stress levels, and whether or not certain diseases are present. While certain gender specific and children's vitamin and mineral formulations are known, these other factors are especially significant in determining proper dosing, yet are largely ignored by many vitamin and mineral supplement manufacturers because of the difficulty in determining how to provide supplements that include ingredients, the type and quantity thereof, that are varied to suit the needs of the individual or the needs of a variety of different classes (or groupings) of individuals.

Accordingly, it is desirable to provide three or more supplements that are to be delivered at sufficiently spaced apart times each day to avoid undesirable interactions between any of the ingredients contained in any of the supplements, to factor in the daily intake of vitamins and minerals that occur through foods based on a person's normal diet, to adjust the quantity of vitamins and minerals in the supplements to provide a Daily Value for the person while helping to prevent severe over-dosing from occurring and, as desired, to factor in the sex, age, lifestyle, health, and any diseases the person may be afflicted with, or to provide a supplement that meets a Daily Value requirement of ingredients for the person and which avoids significant over-dosing of the vitamins or minerals for the person.

Clearly, a method and system for formulating and consuming supplements based on known daily intake values, the compatibility of vitamins and minerals, and optionally individual requirements, whereby the quantity consumed is intended to provide the required daily requirement (Daily Value) while avoiding significant excess intake levels of supplement ingredients above the Daily Value, would be highly beneficial.

Accordingly, there exists today a need for a method and system for formulating and administering supplements based on known daily intake values, the compatibility of vitamins and minerals, and optionally individual requirements that helps to ameliorate the above-mentioned problems and difficulties.

These and other needs in the art are hereby addressed by the devices of the present invention, and methods for the use thereof.

### SUMMARY OF THE INVENTION

In accordance with the present invention, there are provided wearable and/or portable devices for the detection of at least one analyte in a body fluid. Wearable devices can provide a convenient way to monitor various bodily functions, and facilitate prompt treatment of irregular observations such as low blood sugar, high blood pressure, imbalanced blood chemistry, and the like.

As a companion for such wearable analytical devices, portable treatment devices would facilitate treatment of the conditions noted above.

In accordance with certain aspects of the present invention, there are also provided methods for the use of invention devices for the automated administration of essential vitamins, minerals or other nutrients to a subject in need thereof.

In accordance with the present invention, there are also provided body fluid sampling devices.

In certain aspects of the present invention, there are also provided wearable and/or portable devices for the detection of at least one analyte in a body fluid, and the automated administration of essential vitamins, minerals or other nutrients to a subject determined to be in need thereof based on the results provided by the means for detection.

While individuals certainly may go through periods of time when their bodies require higher levels of nutrients for optimal functioning, i.e., during illness or periods of increased stress, the focus of this invention is for the design, manufacture, and managed daily intake of supplements; for formulas which are designed for long term preventative use, and not for temporary therapeutic action. This invention is directed to specifically formulated vitamin and mineral supplements that avoid subjecting individuals to excessively high and potentially harmful doses of substances, while simultaneously preventing negative interactions between supplement ingredients from occurring.

It is to be understood that as ongoing research provides additional data suggesting the inclusion of other vitamins, minerals, or substances as potentially augmenting or otherwise contributing to good health, that the instant method and system is able, as desired, to either modify the ingredients contained in any supplement formulation, or to provide one or more additional supplement formulations that are to be taken at a different time based on the recommendations arising from the ongoing research and which is in agreement with the general teachings of the instant method and system.

It is to be further understood that the instant method and system can be modified to provide supplements as capsules, tablets, pills, dragees, gelatinous capsules, or in liquid form, as desired.

As various embodiments of the instant invention help provide a more elegant solution to the various problems and difficulties as mentioned herein, or which may otherwise exist or occur and are not specifically mentioned herein, and by a showing that a similar benefit is not available by mere reliance upon the teachings of relevant prior art, the instant invention attests to its novelty. Therefore, by helping to provide a more elegant solution to various needs, some of which may be long-standing in nature, the instant invention further attests that the elements thereof, in combination as claimed, cannot be obvious in light of the teachings of the prior art to a person of ordinary skill and creativity.

While the instant invention can be tailored to satisfy the requirements of any desired standard regarding the intake of supplements, for purposes of clarity the description herein refers specifically to Daily Values. For use of the invention with any other standard, the quantities of each required ingredient are adjusted accordingly. It is also possible to vary any of the ingredients in any supplement by omitting certain ingredients or by adding others, as consistent with other standards and in accordance with the compatibility teachings, herein.

### BRIEF DESCRIPTION OF THE FIGURES

FIG. 1A illustrates an exemplary device 10 according to the present invention, held in place about the neck of a subject by collar 1.

FIG. 1B illustrates a back view of collar 1 holding an exemplary device 10 according to the present invention about the neck of a subject.

FIG. 2 illustrates an exemplary device 20 according to the present invention, held in place about the wrist of a subject by band 11.

FIG. 3 illustrates an exemplary device 30 according to the present invention, held in place about the waist of a subject by belt 21.

FIG. 4 illustrates an exemplary device 40 according to the present invention, held in place about the ankle of a subject by wrap 31.

FIG. 5A presents an expanded front view of exemplary devices 10, 20, 30, and/or 40.

FIG. 5B presents an expanded back view of exemplary devices 10, 20, 30, and/or 40, illustrating fluid-containing cartridges 41, 42, 43, 44, 45 and 46.

FIG. 6 illustrates a portable device according to the present invention, comprising lancet cartridge 62, lancet 60, analytical chamber 64, capillary micro-fluidic cartridge 66, wireless transmitter 68, energy storage module 70, LCD indicator 72, foldable USB 74, vitamin and mineral cartridge dispenser 76 and vitamin and mineral injector 78.

FIG. 7 presents an expanded cross-section view of exemplary devices 10, 20, 30, and/or 40, illustrating fluid-containing cartridges 41, 42, 43, 44, 45 and 46.

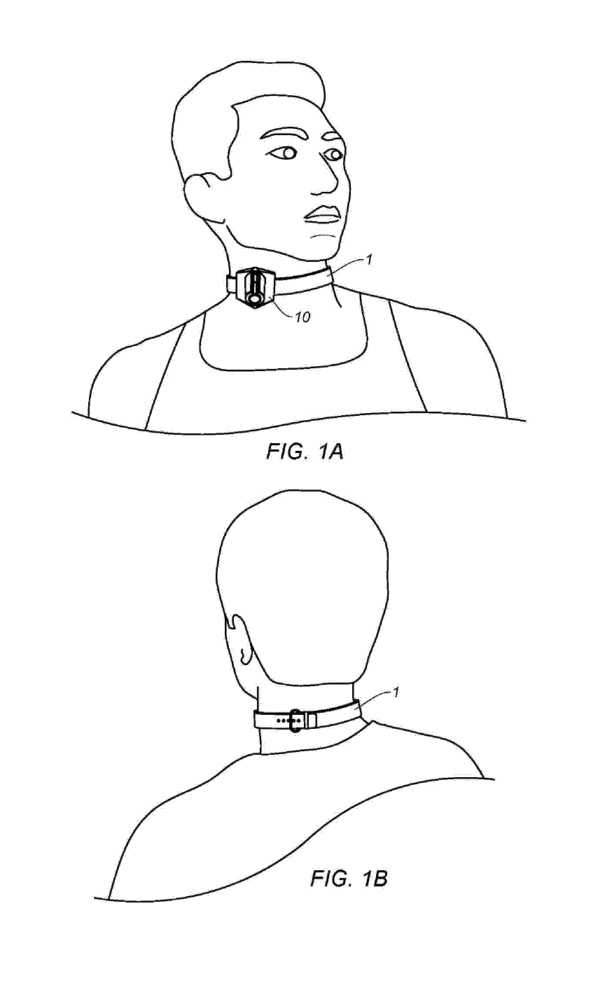

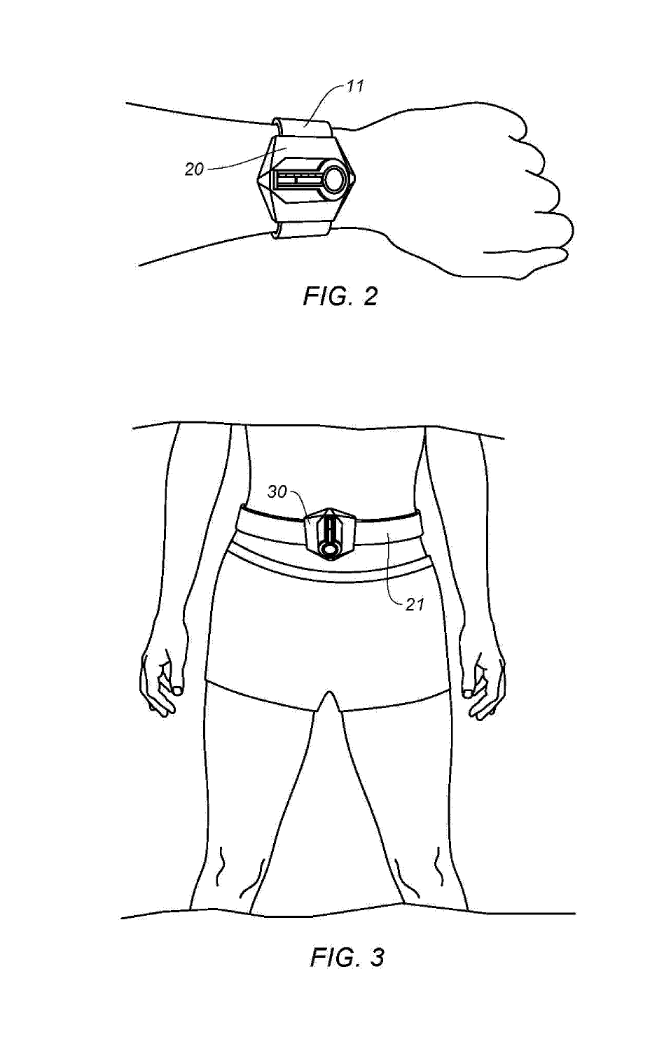

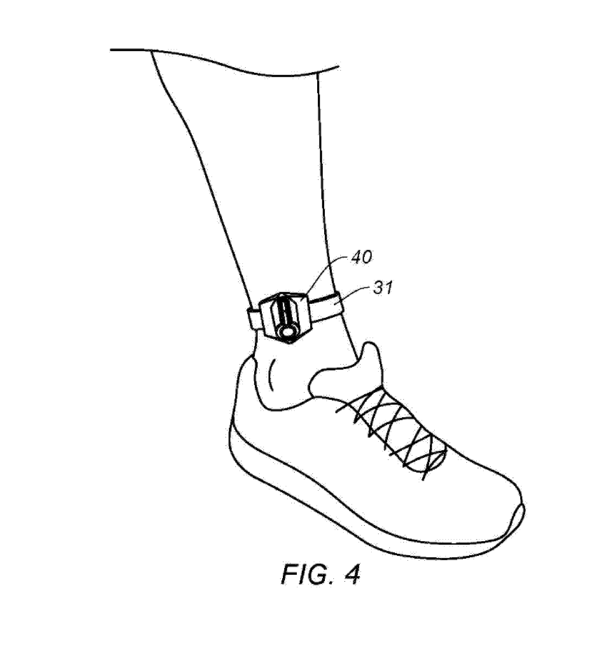

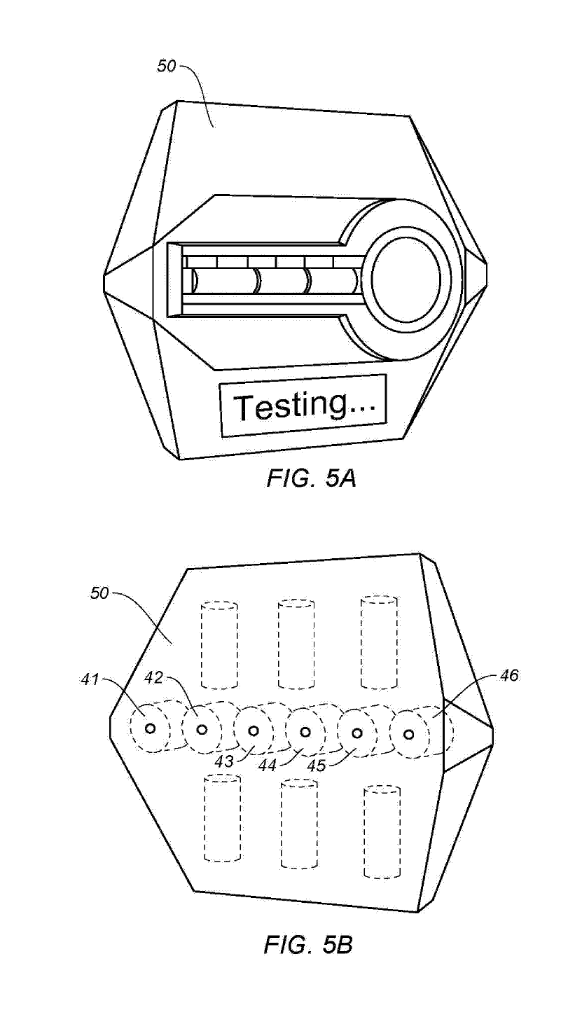

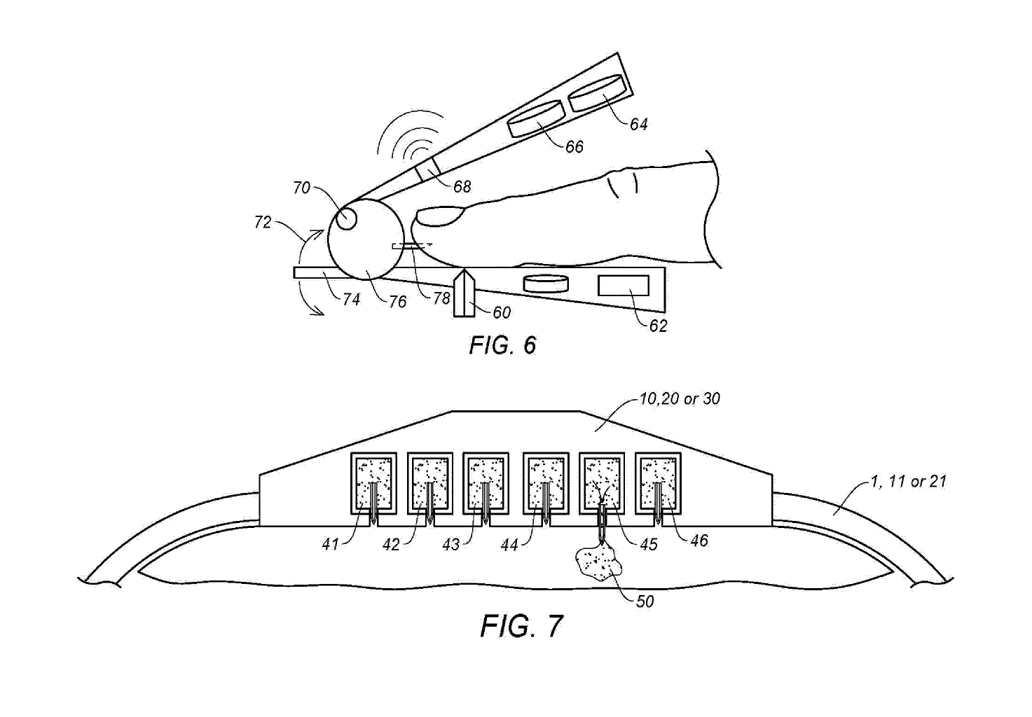

### DETAILED DESCRIPTION OF THE INVENTION

In accordance with the present invention, there are provided wearable and/or portable devices for the detection of at least one analyte in a body fluid, said devices comprising:
(a) means for generating a sample of a body fluid of a subject with whom said device is associated,
(b) a detection zone comprising at least one test element,
(c) means for transferring at least a portion of said sample to said at least one test element,
(d) an indicator or display screen to inform of body fluid analysis; and
(e) means for associating said device in close proximity to a subject in need thereof,
   wherein the device is configured such that the time period between generation of the sample and the application thereof to the test element is substantially instantaneous (e.g., less than 1 second).

As used herein, “means for generating a sample of a body fluid of a subject with whom said device is associated” refers to means for accessing and collecting sufficient body fluid to conduct assays thereon, e.g., a needle, a lancet, and the like.

As used herein, “a detection zone comprising at least one test element” refers to an analytical tool which is useful for evaluation of one or more analytes in a sample of body fluid.

As used herein, “means for transferring at least a portion of said sample to said at least one test element” refers to tubing, capillary tubing, mechanical means, and the like.

As used herein, “an indicator or display screen to inform of body fluid analysis” refers to a read-out of test results obtained with the invention device.

As used herein, “means for associating said device in close proximity to a subject in need thereof” refers to a strap, a clamp, a bracket, an adhesive, and the like.

Devices contemplated herein optionally further comprise a capillary micro-fluidic cartridge blood delivery system.

In accordance with certain aspects of the present invention, the samples of body fluid are generated by means of a skin-piercing element, and the sample enters the detection zone via said capillary micro-fluidic cartridge blood delivery system.

In accordance with certain aspects of the present invention, skin-piercing elements contemplated for use herein may be a lancet, an element that can generate an incision and/or puncture the skin of a user, and the like.

In accordance with certain aspects of the present invention, the lancet further comprises at least one actuator, which is designed to effect a lancet movement of the element for generating the incision and/or puncture for the purpose of generating a sample of body fluid.

In accordance with certain aspects of the present invention, the lancet/skin-piercing element is part of a replaceable cartridge.

In accordance with certain aspects of the present invention, invention devices further comprise a porous substrate which allows for the separation of red blood cells from plasma, and provides accelerated rates of testing for one or more analytes in any given sample of whole blood. In accordance with certain aspects of the present invention, invention devices may further comprise a blood deposit chamber for storing sample blood to be tested.

In accordance with certain aspects of the present invention, invention devices are capable of measuring more than one analyte simultaneously from a single sample.

In accordance with certain aspects of the present invention, invention devices have multiple immunochromatographic pathways (lateral flow pathways) fed by a single sample, thereby rendering the device capable of measuring more than one analyte simultaneously from a single sample.

In accordance with certain aspects of the present invention, invention devices are capable of measuring more than one analyte simultaneously from a single sample by detecting multiple analytes in the same pathway by way of multiple capture antibodies.

In some aspects and embodiments, invention devices may further comprise a digital display, which would indicate the results of blood analysis and suggest administering necessary dosage of highly concentrated vitamin & mineral from the device cartridge chamber to the recipient. The recipient (or caretaker) clicks ‘administer mode’ after viewing the blood test results and the device then injects the recipient with the necessary vitamin and/or mineral, whichever one (or more) is deficient in the recipient's blood. The recipient (or caretaker) might choose NOT to administer the injection upon his or her preference.

A wound received from measuring or subsequent injection can be treated to disinfect the wound, e.g., with an anti-microbial agent (e.g., to prevent infection) and/or a bandage.

In accordance with certain aspects of the present invention, invention devices may further comprise a port for data storage, sharing, charging and/or data transfer. A port can in some embodiments be a USB port, though other types of ports may also be used.

In accordance with certain aspects of the present invention, invention devices may further comprise a wireless transmitter. For example, a wireless transmitter can be used for data storage, sharing, charging and/or data transfer.

In accordance with certain aspects of the present invention, invention devices may further comprise an RFT—Radio Frequency Transmitter, Energy Storage Element/Accumulator.

In accordance with certain aspects of the present invention, invention devices may further comprise a vitamin & mineral storage chamber and/or a vitamin mineral disbursement chamber.

In accordance with another aspect of the present invention, there are provided body fluid (blood) sampling devices comprising:
a skin-piercing element,
a collection zone for receiving body fluid,
a fluid receiving means remotely spaced apart from the collection zone so that body fluid in the collection zone will not contact the fluid receiving means initially, and
means for associating said device in close proximity to a subject in need thereof.

In accordance with certain aspects of the present invention, the collection zone takes up 10 to 500 nl of body fluid in less than 0.5 seconds.

In accordance with certain aspects of the present invention, the fluid receiving means comprises a blood deposit chamber which includes a test chamber/zone for performing the desired analytical protocol.

In accordance with certain aspects of the present invention, the fluid sample from the collection zone is capable of being automatically transported to the fluid receiving means to contact the fluid with the test chamber.

In accordance with still another aspect of the present invention, there are provided wearable and/or portable devices for the detection of at least one analyte in a body fluid, and the automated adjustment of the levels thereof, said device comprising:
(a) means for generating a sample of a body fluid of a subject to whom said device is associated in close proximity thereto, and a deposit chamber for said body fluid,
(b) a detection zone comprising at least one test element,
(c) means for transferring at least a portion of said sample to said at least one test element,
(d) means for determining the level of an analyte of interest,
(e) means for delivering one or more agents which modulate the levels of the analyte of interest, and
(f) means for associating said device in close proximity to a subject in need thereof,
   wherein the device is configured such that the time period between the generation of the sample and the application thereof to the test element is less than 1 second.

In accordance with additional embodiments of the present invention, there are provided methods for the automated administration of essential vitamins, minerals or other nutrients to a subject in need thereof, on an as needed basis, said method comprising associating a device as described herein in close proximity to a subject in need thereof and activating same. Administration can be achieved for example by a needle or other delivery mechanism built into the device. In some embodiments, the position of the needle is moved after each injection so that a subsequent injection is at a different site on the skin compared to the initial injection. In some embodiments, the needle can be rotated in a circle or semi-circle or alternatively positioned along a line at different locations on the skin.

In accordance with yet another embodiment of the present invention, there are provided methods for the automated administration of essential vitamins, minerals or other nutrients to a subject in need thereof, on an as needed basis, said methods comprising:
(1) associating a device in close proximity to a subject in need thereof, said device comprising:
   (a) means for monitoring one or more indicia of body chemistry and/or bodily function of a subject to whom said device is associated in close proximity thereto,
   (b) means for determining whether said one or more indicia of body chemistry and/or bodily function is within an acceptable range,
   (c) means for alerting said subject that it is time to deliver, and/or means for delivering one or more treatments to said subject when one or more indicia of body chemistry and/or bodily function is not within an acceptable range, and
   (d) means for associating said device in close proximity to said subject.
(2) monitoring one or more indicia of body chemistry and/or bodily function of said subject;
(3) identifying one or more indicia of body chemistry and/or bodily function that is not within an acceptable range;
(4) administering one or more suitable treatments to said subject when one or more indicia of body chemistry and/or bodily function is not within an acceptable range; and
(5) repeating steps (2)-(4) at regular time intervals.

As used herein, “means for monitoring” contemplates the use of any available analytical techniques.

As used herein, “indicia of body chemistry” include blood glucose levels, blood oxygen levels, vitamin levels, nutrient levels, and the like.

As used herein, “bodily function” includes pulse, respiration rate, temperature, and the like.

As used herein, “means for determining whether said one or more indicia of body chemistry and/or bodily function is within acceptable range” include comparison to a look-up table, querying a relevant database, and the like.

Devices contemplated for use herein are optionally programmable. The user would input such data as his/her age, and body weight. The device would then calculate the appropriate injection dosage after every blood sample is drawn prior to administering the injection of concentrated vitamins and minerals.

In certain embodiments, the Recipient/Device owner would be able to input his or her personal information into the device (e.g., by inclusion of a hard-drive part/element). Target values can readily be determined by reference to standards provided by the FDA (which offers daily values based on Age and recommends dosages of vitamins and minerals to be consumed on a daily basis).

As used herein, “acceptable range” includes values considered to be “normal”,

As used herein, “means for alerting said subject that it is time to deliver” include an audible alarm, a silent alarm, and the like.

As used herein, “means for delivering one or more treatments” include oral administration, injection, topical application, aerosol, vapor, and the like.

As used herein, “treatments” include vitamin supplements, nutrient supplements, mineral supplements, liquefied herbal supplements, and the like. Exemplary supplements include:
a first supplement formulation, wherein said first supplement formulation includes a first assortment of ingredients, and wherein said first assortment of ingredients includes a plurality of vitamins or minerals or other nutrients, and wherein when said first supplement formulation is ingested by a human being there are no chemical reactions that occur between any of said plurality of vitamins or minerals or other nutrients in said first assortment of ingredients that diminish or adversely impede absorption of said plurality of vitamins or minerals or other nutrients in said first assortment of ingredients into a bloodstream of said human being;
a second supplement formulation, wherein said second supplement formulation includes a second assortment of ingredients, and wherein said second assortment of ingredients includes a plurality of vitamins or minerals or other nutrients, and wherein when said second supplement formulation is ingested by said human being there are no chemical reactions that occur between any of said plurality of vitamins or minerals or other nutrients in said second assortment of ingredients that diminish or adversely impede absorption of said plurality of vitamins or minerals or other nutrients in said second assortment of ingredients into said bloodstream of said human being, wherein said second supplement formulation is different than said first supplement formulation;
a third supplement formulation, wherein said third supplement formulation includes a third assortment of ingredients, and wherein said third assortment of ingredients includes a plurality of vitamins or minerals or other nutrients, and wherein when said third supplement formulation is ingested by said human being there are no chemical reactions that occur between any of said plurality of vitamins or minerals or other nutrients in said third assortment of ingredients that diminish or adversely impede absorption of said plurality of vitamins or minerals or other nutrients in said third assortment of ingredients into said bloodstream of said human being, and wherein said third supplement formulation is different than said first supplement formulation and wherein said third supplement formulation is different than said second supplement formulation;
wherein said system and method includes ingesting said first supplement formulation within a first time-interval range every day, ingesting said second supplement formulation within a second time-interval range every day, and ingesting said third supplement formulation within a third time-interval range every day, wherein said second time-interval range occurs after said first time-interval range, wherein said third time-interval range occurs after said second time-interval range, wherein a first minimum amount of time-separation exists between an end of said first time-interval range and a beginning of said second time-interval range, and wherein a second minimum amount of time-separation exists between an end of said second time-interval range and a beginning of said third time-interval range.
wherein said system and method optionally includes a fourth supplement formulation, wherein said fourth supplement formulation includes a fourth assortment of ingredients and wherein said fourth assortment of ingredients includes a plurality of vitamins or minerals or other nutrients, and wherein when said fourth supplement formulation is ingested by said human being there are no chemical reactions that occur between any of said plurality of vitamins or minerals or other nutrients in said fourth assortment of ingredients that diminish or adversely impede absorption of said plurality of vitamins or minerals or other nutrients in said fourth assortment of ingredients into said bloodstream of said human being, and wherein said fourth supplement formulation is different than said first supplement formulation, and wherein said fourth supplement formulation is different than said second supplement formulation, wherein said fourth supplement formulation is different than said third supplement formulation, wherein said system and method includes ingesting said fourth supplement formulation within a fourth time-interval range every day, and wherein said fourth time-interval range occurs after said third time-interval range.

Said system and method optionally includes a fifth supplement formulation, wherein said fifth supplement formulation includes a fifth assortment of ingredients, and wherein said fifth assortment of ingredients includes a plurality of vitamins or minerals or other nutrients, and wherein when said fifth supplement formulation is ingested by said human being there are no chemical reactions that occur between any of said plurality of vitamins or minerals or other nutrients in said fifth assortment of ingredients that diminish or adversely impede absorption of said plurality of vitamins or minerals or other nutrients in said fifth assortment of ingredients into said bloodstream of said human being, and wherein said fifth supplement formulation is different than said first supplement formulation, and wherein said fifth supplement formulation is different than said second supplement formulation, and wherein said fifth supplement formulation is different than said third supplement formulation, wherein said fifth supplement formulation is different than said fourth supplement formulation, and wherein said system and method includes ingesting said fifth supplement formulation within a fifth time-interval range every day, and wherein said fifth time-interval range occurs after said fourth time interval range.

In certain embodiments of invention systems and methods, the first supplement formulation, or said second supplement formulation, or said third supplement formulation is selected from the group consisting of a capsule, tablet, pill, dragee, gelatinous capsules, and liquid.

In certain embodiments of invention systems and methods, the first supplement first supplement formulation includes vitamins B1, B3, B6, and minerals-iron, copper, molybdenum and iodine.

In certain embodiments of invention systems and methods the second supplement formulation includes vitamins K, H, B2, B5, B6, B9, B12, D3, as well as calcium and the mineral chromium.

In certain embodiments of invention systems and methods, the third supplement formulation includes vitamins A, E, C and minerals magnesium, manganese, selenium, and zinc.

In certain embodiments, invention systems and methods further include a computer operably connected to a memory, and wherein said memory is able to store information specific to any particular person, group, classification or category of people and wherein said system and method includes an algorithm that uses said information to calculate and optimize said first, second and third formulations to provide a desired daily intake of vitamins or minerals or vitamins and minerals to meet the dietary needs of said particular person, group, classification or category of said people. In some embodiments, the algorithm uses the information to determine compatibility between pharmaceutical drugs and one or more nutraceutical (e.g., vitamins & minerals) elements.

In certain embodiments, invention systems and methods comprise a balanced bio-active agent formulation for ingestion by a subject, comprising: a plurality of compatible agent formulations having a plurality of bio-active agent combinations, said plurality of bio-active agents combinations including a first bio-active agent combination having a first combination of vitamins and a first combination of minerals and a second bio-active agent combination having a second combination of vitamins and a second combination of minerals, said agent formulations being based on determined compatibility by and between said bio-active agent combinations, wherein said bio-active agent formulation is selected to eliminate undesirable interactions between any of the ingredients in said first bio-active agent combination and between any of the ingredients in said second bio-active agent combination.

As used herein, “means for mounting said device on a subject in need thereof” include an arm band, an ankle band, a patch, a compression wrap, a neck band, a waist belt, and the like.

Various aspects of the present invention are illustrated by the appended figures. The figures are for illustrative purposes and are not a limitation on any practice of the present invention. It will be understood that variations and modifications can be made without departing from the spirit and scope of the invention.

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

That which is claimed is:

1. A wearable and/or portable device for the detection of at least one analyte in a body fluid, said device comprising:
(a) means for generating a sample of a body fluid of a subject with whom said device is associated,
(b) a detection zone comprising at least one test element,
(c) means for transferring at least a portion of said sample to said at least one test element,
(d) an indicator or display screen to inform of body fluid analysis; and
(e) means for associating said device in close proximity to a subject in need thereof,
wherein the device is configured such that the time period between generation of the sample and the application thereof to the test element is substantially instantaneous (e.g., less than 1 second).

2. The device of claim 1 further comprising a capillary micro-fluidic cartridge blood delivery system.

3. The device of claim 2 wherein said sample of a body fluid is generated by means of a skin-piercing element, and the sample enters the detection zone via said capillary micro-fluidic cartridge blood delivery system.

4. The device of claim 3 wherein said skin-piercing element is a lancet, an element that can generate an incision and/or puncture the skin of a user.

5. The device of claim 4 wherein the lancet further comprises at least one actuator, which is designed to effect a lancet movement of the element for generating the incision and/or puncture for the purpose of generating the sample of the body fluid.

6. The device of claim 4 wherein said lancet/skin-piercing element is part of a replaceable cartridge.

7. The device of claim 1 further comprising a porous substrate which allows for the separation of red blood cells from plasma, and provides accelerated rates for testing for one or more analytes in any given sample of whole blood.

8. The device of claim 1, wherein said device is capable of measuring more than one analyte simultaneously from a single sample.

9. The device of claim 8, wherein said device has multiple immunochromatographic pathways (lateral flow pathways) fed by a single sample, thereby rendering the device capable of measuring more than one analyte simultaneously from a single sample.

10. The device of claim 8 wherein said device is capable of measuring more than one analyte simultaneously from a single sample by detecting multiple analytes in the same pathway by way of multiple capture antibodies.

11. The device of claim 1 further comprising a port for data storage, sharing, charging and/or transfer.

12. The device of claim 1 further comprising a wireless transmitter.

13. The device of claim 1 further comprising an RFT—Radio Frequency Transmitted, Energy Storage Element/Accumulator.

14. The device of claim 1 further comprising a vitamin & mineral storage chamber and/or a vitamin mineral disbursement chamber.

15. A body fluid (blood) sampling device comprising:
a skin-piercing element,
a collection zone for receiving body fluid,
a fluid receiving means comprising a blood deposit chamber which is remotely spaced apart from the collection zone so that body fluid in the collection zone will not contact the fluid receiving means initially, and
means for associating said device in close proximity to a subject in need thereof.

16. The device of claim 15 wherein said collection zone takes up 10 to 500 nl of body fluid in less than 0.5 seconds.

17. The device of claim 15 wherein said fluid receiving means includes a test chamber/zone for performing the desired analytical reaction.

18. The device of claim 15 wherein the fluid sample from the collection zone is capable of being automatically transported to the fluid receiving means to contact the fluid with the test chamber.

19. A method for the automated administration of essential vitamins, minerals or other nutrients to a subject in need thereof, on an as needed basis, said method comprising:
(1) associating a device in close proximity to a subject in need thereof, said device comprising:
(a) means for monitoring one or more indicia of body chemistry and/or bodily function of a subject to whom said device is associated in close proximity thereto,
(b) means for determining whether said one or more indicia of body chemistry and/or bodily function is within an acceptable range,
(c) means for alerting said subject that it is time to deliver, and/or means for delivering one or more treatments to said subject when one or more indicia of body chemistry and/or bodily function is not within an acceptable range, and
(d) means for associating said device in close proximity to said subject.
(2) monitoring one or more indicia of body chemistry and/or bodily function of said subject;
(3) identifying one or more indicia of body chemistry and/or bodily function that is not within an acceptable range;
(4) administering one or more suitable treatments to said subject when one or more indicia of body chemistry and/or bodily function is not within an acceptable range; and
(5) repeating steps (2)-(4) at regular time intervals.
